(19) United States
(12) Patent Application Publication
(10) Pub. No.: US 2025/0174153 A1
(43) Pub. Date: May 29, 2025

(54) Personalized, Non-Verbal Communication to Enhance Mental Health and Detection of Worsening Health Outcomes and Methods, Systems, Devices, and Uses Thereof

(71) Applicant: The Board of Trustees of the Leland Stanford Junior University, Stanford, CA (US)

(72) Inventor: Maheen Adamson, Stanford, CA (US)

(73) Assignee: The Board of Trustees of the Leland Stanford Junior University, Stanford, CA (US)

(21) Appl. No.: 18/839,708

(22) PCT Filed: Feb. 17, 2023
(86) PCT No.: PCT/US2023/062854

§ 371 Date: Aug. 19, 2024

Related U.S. Application Data
(60) Provisional application No. 63/268,154, filed on Feb. 17, 2022.

(51) Int. Cl. G09B 21/00; A61B 5/16; G06F 3/01; G06F 3/0482

(52) U.S. Cl. CPC G09B 21/00; A61B 5/163; G06F 3/013; G06F 3/0482

(57) ABSTRACT

The present disclosure describes systems and methods for non-verbal communication. Aphasia and other speech related disorders can prevent people from communicating effectively or efficiently. Without effective communication, individuals can become depressed, anxious, and/or have worsening health outcomes. Many embodiments described herein allow for communication based on eye tracking of the individual to allow more effective and efficient communication between the individual and a third party, such as a family member or caretaker. Further embodiments are capable of monitoring mental state (e.g., depression and anxiety) and/or providing early detection of health events (e.g., stroke and cognitive decline).

### CROSS-REFERENCE TO RELATED APPLICATIONS

This application claims priority to U.S. Provisional Application Ser. No. 63/268, 154, entitled “Personalized, Non-Verbal Communication to Enhance Mental Health and Prediction of Worsening Health Outcomes and Methods, Systems, Devices, and Uses Thereof” to Maheen Adamson, filed Feb. 17, 2022, the disclosure of which is incorporated herein by reference in its entirety.

### FIELD OF THE INVENTION

The present invention relates generally to communication methods for non-verbal individuals and uses thereof; more particularly, methods that allow individuals with communication disorders such as aphasic individuals to communicate more effectively, which also allow for the early detection of adverse health outcomes, such as stroke.

### BACKGROUND OF THE INVENTION

Approximately one-third of 750,000 ischemic and hemorrhagic strokes per year (˜225,000) in the US lead to aphasia, a communication disorder, in which 40% of the patients suffer from post-stroke severe disabilities. (See e.g., A Towfighi, et al. Poststroke depression: a scientific statement for healthcare professionals from the American Heart Association/American Stroke Association. Stroke, 2017. Am Heart Assoc.; the disclosure of which is incorporated herein in its entirety.) Fifteen percent of patients below 65 years old experience aphasia after their first stroke, which increases to 43% in patients above 85 years old. (See e.g., Ellis, C., et al. The one-year attributable cost of poststroke aphasia. Stroke, 2012—Am Heart Assoc.; the disclosure of which is incorporated herein in its entirety.) More importantly, in this type of communication disorder where you lose your ability to speak and communicate, the incidence of depression in post-stroke aphasia is estimated to be 52-70% and is higher than in stroke survivors without aphasia. Overall, aphasia adds to stroke-related care costs, above the cost of stroke alone, (˜$1700 addition/patient). (See e.g., Kroll, A. & Karakiewicz, B. Do caregivers' personality and emotional intelligence modify their perception of relationship and communication with people with aphasia? Int. J. Lang Commun Disord, 55: 661-677; the disclosure of which is incorporated herein in its entirety.) Patients with aphasia experience longer length of hospital stays, greater morbidity, and greater mortality. Within the stroke population, stakeholders such as patients, caregivers, neurologists, speech pathologists all agree (personal interviews) that there are few resources available to improve communication for an aphasic patient post-stroke leading to prolonged depression. In addition to the inability to communicate, cognitive and motor control issues, patients also disconnect from their caregivers (also known as communication partners) who play a crucial role in the rehabilitation of an aphasic patient.

Although there is a plethora of research and clinical application directed towards patients with aphasia, and others with communication disorders there is a dearth of solutions available in both acute and outpatient settings that lead to better psychiatric outcomes. Treatment solutions currently available in the market typically do not involve the caregiver nor do they provide alternative methods of communication that do not rely on speech. Additionally, current solutions cannot be aligned with speech therapy provided to the patient improve communication. Additionally, communication devices that incorporate eye tracking, these solutions incorporate eye tracking using indiscrete and cumbersome eye trackers attached to special devices for severely injured and/or handicapped adults.

Many of the currently available solutions are borrowed from developmental disorders such as autism, where the cognitive abilities are developing in a very different pattern than what is seen in aphasia or other adult communication disorders (e.g., someone intubated in an ICU or someone with throat cancer where these cognitive abilities are more intact). Additionally, although there is an agreement in the clinical community that communication partners/caregivers play a crucial role in the rehabilitation of interpersonal communication with an aphasic or non-communicative patient, most solutions do not involve caregivers' perspective and rely solely on the interaction of the patient with the cloud. This hampers not just communication but monitoring and assessment for future health problems. (See e.g., Van Dam, Levi, et al. “Can an Emoji a Day Keep the Doctor Away? An Explorative Mixed-Methods Feasibility Study to Develop a Self-Help App for Youth With Mental Health Problems.” Frontiers in Psychiatry, vol. 10, August 2019, p. 593 Int. J. Lang Commun Disord, 55: 661-677; the disclosure of which is incorporated herein in its entirety.) The inability to address these gaps that highlight the absence of communication and social engagement between patient and the communication partner/caregiver has led to very few effective treatments available in acute and outpatient settings for this and other populations with communication disorders.

### SUMMARY OF THE INVENTION

Methods, systems, and devices for personalized, non-verbal communication to enhance mental health and detection of worsening health outcomes are disclosed.

In some aspects, the techniques described herein relate to a method including providing a device to an individual, where the device includes a display and an input device, where the display provides a set of stimuli and where the input device is capable of tracking an eye of the individual, where the input device monitors focus of the individual and where the individual's focus on a stimulus in the set of stimuli provides a signal to select that input.

In some aspects, the techniques described herein relate to a method, where the selection of the stimulus transmits a request to a caretaker.

In some aspects, the techniques described herein relate to a method, where the device is capable of detecting changes in focus which are indicative of a mental state.

In some aspects, the techniques described herein relate to a method, where the mental state is selected from depression, anxiety, stress, and fatigue.

In some aspects, the techniques described herein relate to a method, where the device is capable of detecting a health event and/or early detection of a health event.

In some aspects, the techniques described herein relate to a method, where the health event is selected from stroke and cognitive decline.

In some aspects, the techniques described herein relate to a device, where each stimulus in the set of stimuli is displayed as an icon.

In some aspects, the techniques described herein relate to a device, where the set of stimuli include at least one of personal needs, mood, food, and drink.

In some aspects, the techniques described herein relate to a device, where at least one stimulus in the set of stimuli represents a hierarchical menu, where selection of the at least one stimulus provides a second set of stimuli with more specificity.

In some aspects, the techniques described herein relate to a device for non-verbal communication including a display to provide a set of stimuli to an individual, and an input device capable of tracking an eye of the individual, where the input device monitors focus of the individual and where the individual's focus on a stimulus in the set of stimuli provides a signal to select that input.

In some aspects, the techniques described herein relate to a device, further including a wireless communication device capable of sending information to another device.

In some aspects, the techniques described herein relate to a device, where each stimulus in the set of stimuli is displayed as an icon.

In some aspects, the techniques described herein relate to a device, where the set of stimuli include at least one of personal needs, mood, food, and drink.

In some aspects, the techniques described herein relate to a device, where at least one stimulus in the set of stimuli represents a hierarchical menu, where selection of the at least one stimulus provides a second set of stimuli with more specificity.

In some aspects, the techniques described herein relate to a system for non-verbal communication including a patient device, including a display to provide a set of stimuli to a patient, and an input device capable of tracking an eye of the patient, where the input device monitors focus of the individual and where the individual's focus on a stimulus in the set of stimuli selects that stimulus, and a caretaker device, including a display to provide information to a caretaker, and an input device capable of accepting input from the caretaker, where a request from a patient is displayed on the display and the caretaker can provide input via the input device to acknowledge a request, where the selection of a stimulus from the patient device sends a request to the caretaker device.

In some aspects, the techniques described herein relate to a system, where the patient device and the caretaker device each further include a wireless communication device capable of sending and receiving information to each other.

In some aspects, the techniques described herein relate to a system, where each stimulus in the set of stimuli is displayed as an icon.

In some aspects, the techniques described herein relate to a system, where the set of stimuli include at least one of personal needs, mood, food, and drink.

In some aspects, the techniques described herein relate to a system, where at least one stimulus in the set of stimuli represents a hierarchical menu, where selection of the at least one stimulus provides a second set of stimuli with more specificity.

In some aspects, the techniques described herein relate to a system, where the caretaker can provide input via the input device to mark a request as complete.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying drawings where:

FIG. 1 provides an example of a patient device for non-verbal communication in accordance with various embodiments.

FIGS. 2A-2B provide examples of a caretaker device for non-verbal communication in accordance with various embodiments.

FIG. 3 provides an exemplary method for using a non-verbal communication system in accordance with various embodiments.

FIG. 4 provides examples of emojis for communication mood in accordance with various embodiments.

FIG. 5 provides an exemplary method for optimizing a device for non-verbal communication system in accordance with various embodiments.

FIG. 6 illustrates a block diagram of components of a processing system in a computing device that can be used for non-verbal communication in accordance with various embodiments.

FIG. 7 illustrates a network diagram of a distributed system that can be used for non-verbal communication in accordance with various embodiments.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

Turning now to the drawings, methods, systems, and devices for personalized, non-verbal communication to enhance mental health and/or early detection of worsening health outcomes are illustrated. Many embodiments described herein provide a multi-layer communication-diagnostic method using eye tracking technology, speech therapy stimuli used currently in clinical setting and standardized assessment tools for depression, anxiety, sleep, cognitive decline and fatigue. Because aphasia in stroke, intubated patients or patients with throat cancer leads to loss of speech and/or motor control this method must include (in addition to voice commands and touch screen) a non-speech and non-limb-based communication method that is both accurate, efficient and easily monitored with measurable outcomes (e.g., reduced depression). Additional embodiments serve as a platform for monitoring and assessment of eye movements along with physiological monitoring to capture early signs of future health problems.

### Systems for Non-Verbal Communication

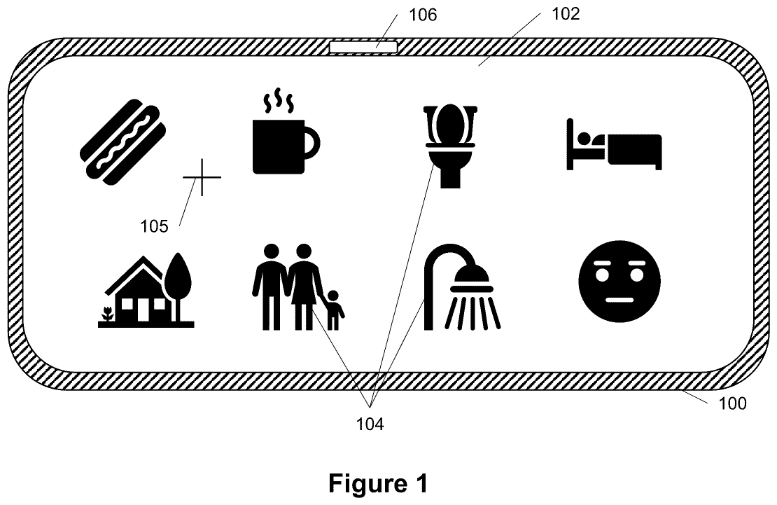

Turning to FIG. 1, many embodiments provide for a device 100 that is capable of receiving input from a user with reduced ability to communicate, such as aphasia. Such devices can be computing devices (e.g., devices including a processor and memory, where the processor is capable of performing certain actions based on instructions contained within the memory). Many embodiments include a display 102 that is capable of visually displaying one or more stimuli and/or outputs.

In many embodiments, display 102 is configured to display one or more stimuli 104 to allow an individual (e.g., a patient) to communicate to a caretaker, such as a doctor, nurse, social worker, family member, friend, etc. These stimuli can include requests for basic needs, personalized stimuli, and assessments for mental condition (e.g., depression, anxiety, sleep, falls, fatigue, stroke, cognitive health, etc.) The stimuli can be displayed as icons and/or text to indicate a need or desire for the patient. Various embodiments display a set of icons that are constant, while other embodiments may update icons to comply with schedules, such as periodic requests for patient input about mental health. Some embodiments display a cursor 105 or other pointer on the display 102. A cursor 105 can assist a patient in understanding where they are looking on the screen and make sure the correct stimulus is selected.

Various embodiments utilize a hierarchical menu for stimuli, such that selection of, or response to, one stimulus opens an additional set of icons to allow for specific selections by the patient. For example, a selection of “food” may open a secondary menu of food items, such as preferred or favorite items, while selecting “drink” may allow selection of coffee, tea, water, soda, etc. Additionally, personal needs may include requests for medication, family, hobbies, prior career, and interests.

Additionally, certain stimuli can be used in speech therapies, including stimuli from curriculum usually used by the speech therapist/pathologist that can be individualized for the patient and then digitized for practice on a device 100.

Additional embodiments include an input device 106 to allow a user to select a stimulus and/or otherwise interact with the device 100. As noted previously, aphasia can be caused by stroke and/or other conditions that may also cause reduced physical ability, mobility, and/or another ailment. As such, many embodiments include an input device 104 that can allow for input based on non-tactile input (e.g., eye motion and/or eye tracking). Such tracking can be accomplished with existing eye tracking technology (e.g., cameras, sensors, etc.). Certain embodiments are further enabled with components, including (but not limited to) the AR kit of Apple iPads® and/or any other similar product to improve the ability to track eyes and/or eye motion.

Using a device 100 can allow an individual to communicate to a caretaker (e.g., doctor, nurse, therapist, family member, friend, etc.) by providing an intuitive as functional system to receive patient inputs and responses. In many embodiments, when an input or response is received from a patient, a request is transmitted to the caretaker via a caretaker device. A caretaker device may be similar to a patient device (e.g., device 100) including a display 102 and input device 106. However, eye tracking capabilities may not be necessary, as a caretaker is likely to be mobile and capable of tactile input.

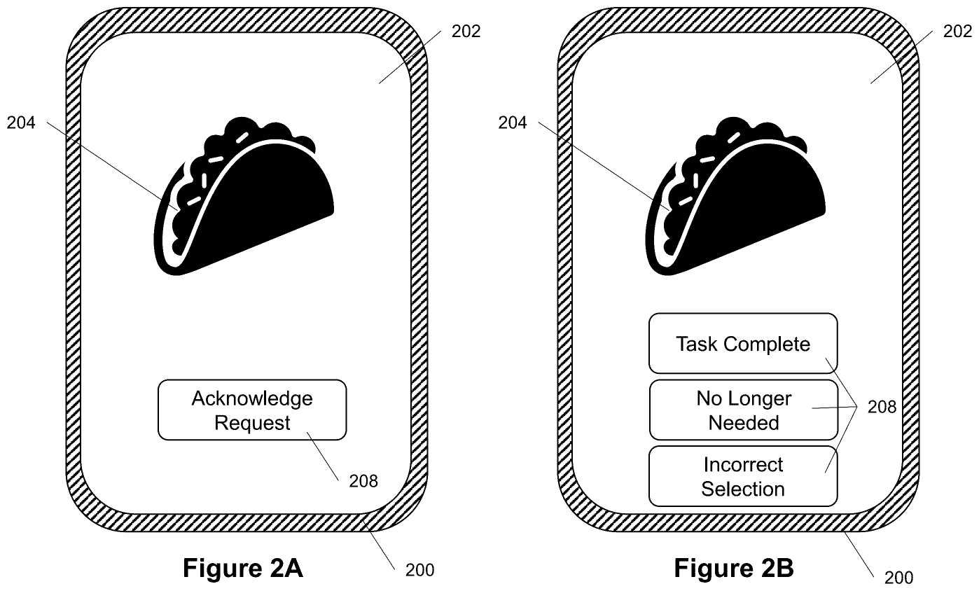

Turning to FIG. 2A, when a caretaker device 200 receives a request from a patient, such as a request for food, drink, etc., the request can be displayed as an icon 204 and/or another item on a caretaker's display 202. Additional details about the request, including (but not limited to) time of request, time since request, individual making the request, and/or any other relevant details can be provided with the request. Further embodiments include an option box 208 for the caretaker to acknowledge such request. Turning to FIG. 2B, when a caretaker fulfills a request, the option box 208 may change to mark fulfillment of the request (e.g., “Task Complete”). In certain embodiments, additional options may exist to mark completion of the request, including “Incorrect Selection,” “No Longer Needed,” and/or any other option that may identify the completion of the request. In some embodiments, proximity to the patient may be required to acknowledge completion. Such proximity can be identified via any relevant mechanism to identify a caretaker within a specified distance to the patient, such as by connection via Bluetooth communication, nearfield communication, infrared communication, GPS position of a caretaker device, and/or any other possible way to identify the proximity between a caretaker and patient.

The specifics for a caretaker device may differ for different environments and/or may form an open-or closed-loop system between a patient device and caretaker device. For example, when therapy and/or medical oversight is active, certain information (e.g., mood, medication requests, etc.) may be routed to a physician and/or therapist caretaker, while personal needs (e.g., food, drink, etc.) may be routed to a nurse and/or orderly caretaker. Such information can be securely transmitted via cloud-based and/or local-network-based systems.

Many embodiments herein are capable of identifying many pieces of data that can identify worsening conditions for action by a medical caretaker. Many embodiments can gather information either automatically or by manual input, including (but not limited to) demographic information (e.g., age, gender/sex, use of vision correction, type of affliction (e.g., stroke, injury, etc.), and other relevant medical history). Some embodiments collect data based on usage of devices, including (but not limited to):
Time for Selection (TFS) Time from when eye contact is made on the Stimuli screen to reasonable eye lock on chosen stimuli
Accuracy in Selection (AIS) Accuracy of eye selection of stimuli compared to actual stimuli wanted
Caregiver Response Time (CRT) Amount of time between sending of stimulus request to the acknowledgment of request on Caregiver device.
Degradation in Selection Time (DST) Difference in (TFS) over a given time.
Head Position Vector (HPV) Nodding off indication.
Fixation Rate (FR)
Blink Rate (BR)
Dwell Time
Fixation and gaze points
Time to first fixation
Fixation sequences
Revisits
First fixation duration
Average fixation duration
Speed of movement
Acceleration of movement
   Environmental conditions may further be collected or obtained in some systems, including (but not limited to) time of day, date, environmental factors (e.g., brightness, lighting type, etc.), hardware type (e.g., make, model, software version, hardware version, firmware version), communication speed (e.g., data upload/download), time to response from communication partner, head position (e.g., upright, tilted). To mitigate imaging or tracking issues due to lighting, certain embodiments include an additional light source to assist in illuminating a subject or user. Lighting can be infrared, visual, or any other wavelength(s) that is non-ionizing or non-damaging. Additional data can be included during or after use, such as user comfort, user mood or feelings, usability, functionality, and/or any other user feedback. Certain embodiments collect physiological data, including (but not limited to) heart rate, sleep activity, gait and/or motion, and other collectable physiological data.

Data can be task-specific, such as from providing a task to a user, then collecting pieces of data (e.g., speed, linger time, etc.) which can be obtained. As an example, for classification problems, F1-score can be used, which is the combination of precision and recall. Adjustments to the default metric will be made due to the “consequences” of “false negatives”. Once the tasks are defined, quality thresholds can be defined for given metrics. For example, faster times for transfer of object stimuli to communication partner device, higher prediction of accuracy will lead to faster response from communication partner and translate to better assessment scores for various states, such as (but not limited to) depression, anxiety, fatigue, and and/or any other relevant state. In certain embodiments, synthetic data can be obtained from other sources, such as imputation and/or open-source.

Further embodiments validate data based for real-world scenarios, corner-cases (e.g., missing data), and validate for lighting conditions, correct balancing, realistic camera input. Once validated, metrics can be defined with respect to this “effective & balanced” dataset.

Various embodiments implement machine learning systems to assess user's action and/or intention for input. For example, certain embodiments implementing machine learning can predict observable events (e.g., blinks, fixation, vergence, segment out oculomotor behavior, etc.). Further embodiments can include regression head, to determine one or more of: time before the event, prediction confidence scores, time taken to deliver the stimuli, to complete the task (e.g., bring water to patient as requested) and accuracy of the task (e.g., was it tea, coffee, or water?).

The amount and types of data can be stored locally (e.g., in a memory of a device) or remotely, such as cloud-based or other network connected storage device (e.g., server).

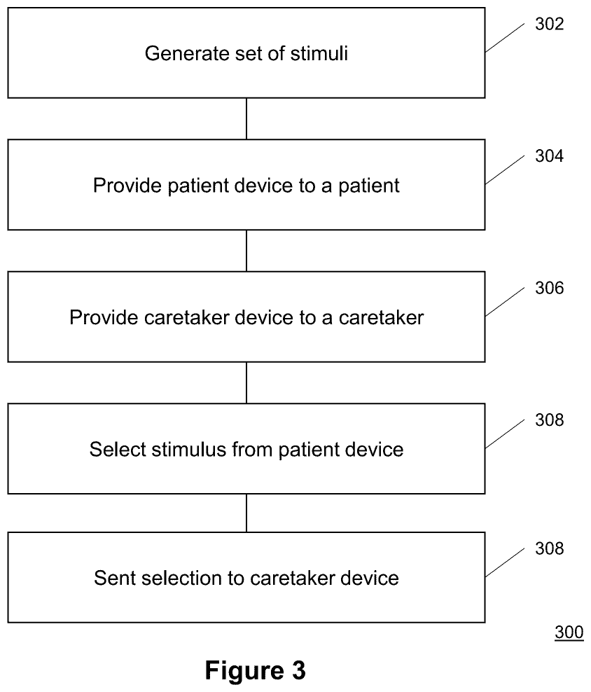

FIG. 3 provides an exemplary method 300 for using a device in accordance with many embodiments. As illustrated, at 302, many embodiments generate a set of stimuli to be displayed on a patient device. Patient devices are described elsewhere herein, including the exemplary device 100 of FIG. 1. The set of stimuli can include particular needs, wants, etc. of a patient and/or a caretaker. This set may further be specific to the setting of where the patient will be using the device, such as at home or in a care facility (e.g., in-patient, out-patient, acute care, etc.) The positions of the stimuli in the set of stimuli may also be prioritized for likelihood of use, personally preferred location, and/or location that the patient may be able to view (e.g., for blindness, hemispheric issues, etc.) Additionally, sizes of each stimuli may be altered for low-vision issues in the patient. Once the stimuli are generated, these stimuli can be sent to a patient device.

At 304, many embodiments provide the patient device to the patient. Similarly, a caretaker device may be provided to a caretaker at 306. Such caretaker devices are described elsewhere herein, including the exemplary device 200 of FIGS. 2A-2B. In many embodiments, a caretaker may already possess a caretaker device, such as an in-patient or acute-care facility, where the caretaker provides care to multiple individuals.

At 308, a patient selects a stimulus on the patient device. Such selection methodologies are described herein and can include hierarchical structuring. This stimulus can be sent to a caretaker device at 310. As noted herein, the communication can occur via many methods and/or routings, such as when multiple caretakers are responsible for different aspects of the patient's care (e.g., medicine, food, drink, etc.). The receiving caretaker can acknowledge the request as well as mark completion of the request when performing the request.

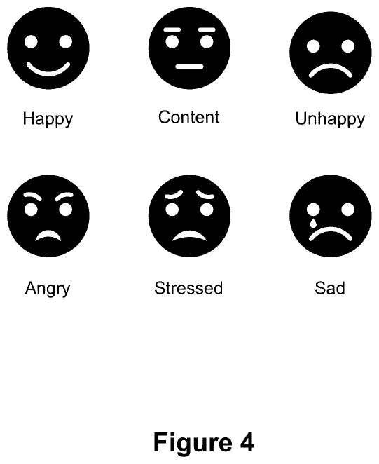

It should be noted that method 300 is merely an example and is not meant to be exhaustive and/or comprehensive of all possible embodiments. As such, certain embodiments may add features, remove features, omit features, repeat features, perform features simultaneously, perform features in a different order, and/or any other combination of possible. For example, certain stimuli may be induced to understand a patient's wellbeing, satisfaction, mood, and/or any other self-assessment. Such stimuli may be induced by a caretaker or on a periodic schedule and will not submit an actionable request to a caretaker. For instance, certain embodiments use questionnaires (e.g., Geriatric Depression Scale), which can be conducted by the communication partner, with digitized versions of mental health assessments done multiple times daily. (See e.g., Sheikh, J. I., & Yesavage, J. A. (1986). Geriatric Depression Scale (GDS): Recent evidence and development of a shorter version. Clinical Gerontologist, 5, 165-173; the disclosure of which is incorporated herein in its entirety.) A recent development has led to a new form of measurement, ecological momentary assessment (EMA), where one can repeatedly assess the behavior of an individual in their natural environment using emoji or other digital apps. Many embodiments analyze the continuously monitored data and link it with the frequency and accuracy of completed tasks, time to complete the tasks, and general health of the patient. Additional assessments include standardized assessments for depression (GDS), fatigue (Flinders' Fatigue Scale), stress (Perceived Stress Scale), anxiety (Beck Anxiety Index), and cognitive decline (Montreal Cognitive Assessment (MoCA) by patient and caregiver. However, certain embodiments utilize emojis to gauge anxiety, stress, mood, fatigue and cognitive difficulty; examples of which are illustrated in FIG. 4.

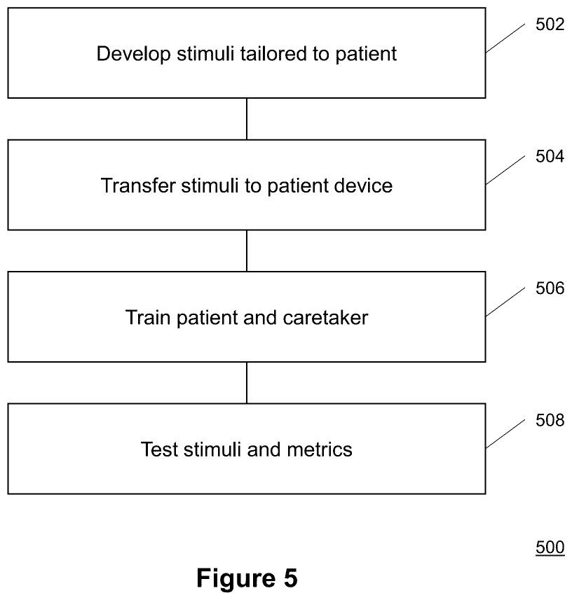

Various embodiments also optimize the stimulus-response methodology for a particular environmental scenario, including (but not limited to) an acute in-patient setting, an outpatient/in-home setting, and/or combined settings. FIG. 5 illustrates an exemplary method 500 for training a stimulus-response methodology. Such methodology allows for training individuals how to use a system but also to calibrate and/or optimize parameters to the inputs and responses of an individual. At 502, many embodiments develop stimuli tailored to the individual. As noted previously, such stimuli can be for personal needs, wants, care, etc. for the individual as well as the particular environment (e.g., outpatient, inpatient, etc.) for the individual.

The stimuli can be transferred to a patient device at 504. Transferring stimuli can include uploading, selecting from a menu, and/or any other method to allow the patient device to display the stimuli. In many embodiments, the stimuli are displayed as icons, lists, or any/other method to display the stimuli. In certain embodiments, the stimuli are further displayed in a preferred position for the individual, such as based on priority, personal preference, likelihood of or amount of use, and/or any other reason. Further embodiments display stimuli on only part of a screen, such as when a patient only has the ability to see part of a display, such as through blindness and/or hemispheric issues with the brain. In some embodiments, the transferring includes applying initial calibrations, metrics, and/or other optimization metrics.

At 506, the patient can be trained to use the respective devices. Such training can include directing an individual to select a stimulus using their eyes (such a request can be a considered a response to a stimulus). Selection of a stimulus can occur based on the eye tracking, such as dwell time on a stimulus, blinking, or a specific pattern of blinks. The actions to select a stimulus can vary for an individual based on the position of the stimulus.

The stimuli-response system can be tested with the individual to identify a metric, such as optimization, calibration, etc. for the individual at 508. For example, depending on severity of a patient's condition, a response may be recorded inadvertently because of slower movement, while other patients may allow for shorter times or a different pattern of blinks to select a stimulus. Such metrics can be updated based on the individual's preferences or abilities. Additionally, specific selection actions (e.g., dwell, blinks, etc.) can be changed based on efficacy for the individual.

It should be noted that various features of method 500 are merely exemplary and are not comprehensive with all embodiments. Additionally, certain features may be added, which are not explicitly described in method 500, while some illustrated features may be omitted, performed in a different order, repeated, and/or performed at the same time without straying from the scope of all embodiments described herein.

### Predicting Mental State or Future Health Events

The use of eye tracking, particularly initial gaze orientation and gaze maintenance has been previously used to detect mood changes in post-stroke aphasic patients. (See e.g., Ashaie, Sameer A., and Leora R. Cherney. “Eye Tracking as a Tool to Identify Mood in Aphasia: A Feasibility Study.” Neurorehabilitation and Neural Repair, vol. 34, no. 5, May 2020, pp. 463-71; the disclosure of which is hereby incorporated by reference in its entirety.) However, certain embodiments can collect the individual eye gazing metrics that can detect early symptoms of other disorders. In fact, previous work has shown promise for the utility of eye tracking as a diagnostic and therapeutic index of language functioning in patients with anomia (progressive naming impairment). (See e.g., Ungrady, Molly B., et al. “Naming and Knowing Revisited: Eye tracking Correlates of Anomia in Progressive Aphasia.” Frontiers in Human Neuroscience, vol. 13, October 2019, p. 354; the disclosure of which is hereby incorporated by reference in its entirety.) Applying previously obtained findings of similar eye tracking measures to our data collected can help convert our communication method into a diagnostic tool for post-stroke aphasic patients or those who have a communication disorder who may be on the path of further health decline.

As such, many embodiments utilize an algorithm based on eye tracking metrics to predict early symptoms of other health problems such as anxiety, stress, cognitive decline and/or early detection of stroke. To achieve this, various embodiments use known metrics (e.g., initial gaze, gaze orientation, gaze maintenance, etc.) to guide in capturing preclinical symptoms of depression, fatigue, anxiety, and cognitive decline and capture early detection of neurological events like stroke. The continuous monitoring, use of standardized stimuli, and the power of eye tracking metrics will enable such embodiments to deliver beneficial outcomes (e.g., reduced depression) via increased communication and social engagement. Further embodiments integrate heart rate and sleep components from additional applications. For example, data (eye tracking metrics, assessments, emojis etc.) can be continuously exchanged between a patient and a care team while being safely stored in the cloud, in accordance with many embodiments. Also, displays that a model set algorithm can be utilized in the cloud, continuously matching the information with baseline data collected, to inform the care team. In such embodiments, each person has personalized access to the dashboard. In addition, in the scenario when an event like a stroke occurs, alerts can also be sent to caretakers and/or medical providers. Additional embodiments can also access a patient's electronic health records in order to provide direct assessments on the fast-track recovery to primary providers and rehabilitation care team and alerts.

To detect and/or predict mental state and health events, many embodiments define priority, monitoring, delivery and alert system in the model set algorithm. Such definition can include assessing depression, mood, anxiety and compare with baseline scores; assessing eye tracking metrics with baseline eye tracking obtained; assessing heart rate and sleep activity with assessments and eye tracking activity; and monitoring when such certain metrics pass a threshold based on baseline scores or a single event occurs like a stroke. Various embodiments deliver to caregiver and provider daily and alert when the threshold is passed.

### Network and Software Implementations

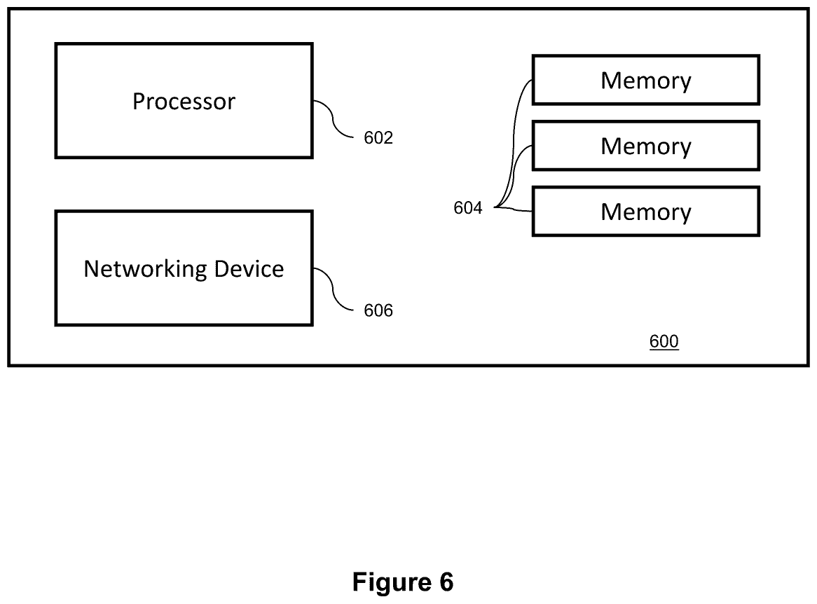

Processes that provide the methods and systems for personalized, non-verbal communication to enhance mental health and detection of worsening health outcomes in accordance with some embodiments are executed by a computing device or computing system, such as a desktop computer, tablet, mobile device, laptop computer, notebook computer, server system, and/or any other device capable of performing one or more features, functions, methods, and/or steps as described herein. The relevant components in a computing device that can perform the processes in accordance with some embodiments are shown in FIG. 6. One skilled in the art will recognize that computing devices or systems may include other components that are omitted for brevity without departing from described embodiments. A computing device 600 in accordance with such embodiments comprises a processor 602 and at least one memory 604. Memory 604 can be a non-volatile memory and/or a volatile memory, and the processor 602 is a processor, microprocessor, controller, or a combination of processors, microprocessor, and/or controllers that performs instructions stored in memory 604. Such instructions stored in the memory 604, when executed by the processor, can direct the processor, to perform one or more features, functions, methods, and/or steps as described herein. Any input information or data can be stored in the memory 604—either the same memory or another memory. In accordance with various other embodiments, the computing device 600 may have hardware and/or firmware that can include the instructions and/or perform these processes.

Certain embodiments can include a networking device 606 to allow communication (wired, wireless, etc.) to another device, such as through a network, near-field communication, Bluetooth, infrared, radio frequency, and/or any other suitable communication system. Such systems can be beneficial for receiving data, information, or input from another computing device and/or for transmitting data, information, or output (e.g., risk score) to another device.

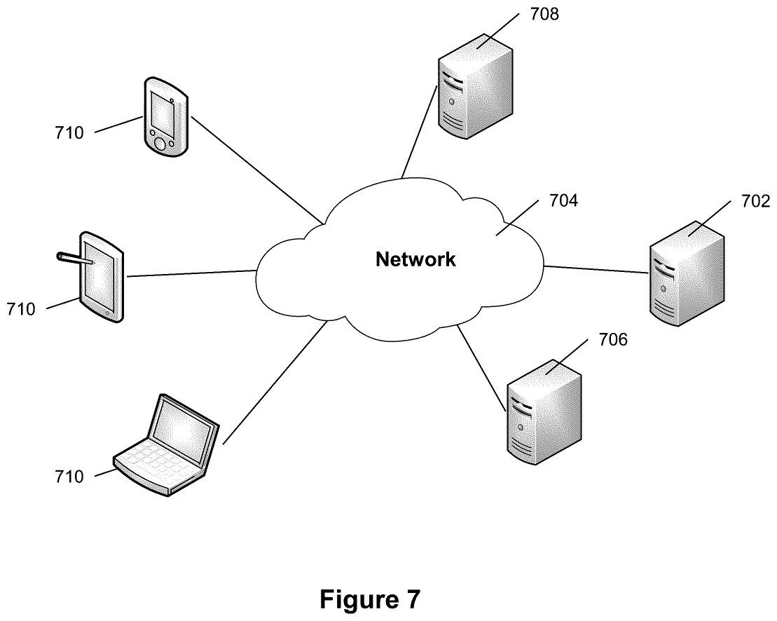

Turning to FIG. 7, an embodiment with distributed computing devices is illustrated. Such embodiments may be useful where computing power is not possible at a local level, and a central computing device (e.g., server) performs one or more features, functions, methods, and/or steps described herein. In such embodiments, a computing device 702 (e.g., server) is connected to a network 704 (wired and/or wireless), where it can receive inputs from one or more computing devices, including data from a records database or repository 706, data provided from a local computing device 708, and/or any other relevant information from one or more other remote devices 710. Once computing device 702 performs one or more features, functions, methods, and/or steps described herein, any outputs can be transmitted to one or more computing devices 706, 708, 710 for entering into records, taking personal and/or medical action. Such actions can be transmitted directly to a medical professional (e.g., via messaging, such as email, SMS, voice/vocal alert, another computing device) for such action and/or entered into medical records.

In accordance with still other embodiments, the instructions for the processes can be stored in any of a variety of non-transitory computer readable media appropriate to a specific application.

### EXEMPLARY EMBODIMENTS

Although the following embodiments provide details on certain embodiments of the inventions, it should be understood that these are only exemplary in nature, and are not intended to limit the scope of the invention.

### Example 1: Building a System

### Methods

1. Choose a population/setting: Typical aphasia patient post stroke (Expressive aphasia & Progressive Aphasia). Choose severity based on Western Aphasia Battery scores (mild, moderate) because comprehension must be intact and object recognition mostly not affected. We will test a range of severity scores. Acute setting for post stroke patients to start using the device, then home and rehabilitation setting.
a) Intubated patients in acute setting who cannot use speech.
b) Throat cancer patient's in-patient or out-patient
c) Other adults with communication disorder

2. Create basic stimuli for population (content creation with neurologist and speech pathologist):
a) Test visual load/linguistic and symbol complexity (test simple to complex objects)
b) Use real pictures, large size on screen, high resolution
c) Establish whether response can be consistent with Y/N or do we need a few more categories.
d) Establish basic need categories for stimuli.
e) Establish outcome measures surveys (self-report) for caregiver (standardized forms) and patient (non-verbal e.g., emojis)

3. Transfer Stimuli to Device (individual/patient) and establish tracking paths for object recognition, selection, recording and transfer to another device (communication partner/assistant).
a) Test the length, frequency and other tracking metrics for each stimuli to be transferred with accuracy to another device.
b) Test the time and accuracy it takes for each stimuli (request) to be completed by the caregiver/assistant.
c) Test the validity of these stimuli and the metrics to be recorded in the cloud for the two-way interaction between patient and caregiver.

4. Develop the system for collection, monitoring and analysis of model sets in the cloud
a) establish transmitting eye-tracking metrics associated with communication behavior of the individual (patient) during a time period including object recognition;
b) receiving dataset characterizing transfer of the object image to caregiver's (another individual) device during this the time period;
c) receiving a dataset for completion of the task during this time with specific time stamps;
d) generating an outcome dataset upon retrieving responses provided by the individual to all self-report forms either depicted by emojis or conducted by caregiver at specific time points;
e) generating a continuous report from the passive eye tracking dataset derived from the log of use dataset, completion of task dataset and all daily eye tracking metrics;
f) generating a daily report summarizing the communication successes, mental health state of the individual (patient), based on the responses on the outcome surveys;
g) generating a daily report summarizing eye tracking metrics, and relating it to the various health outcomes for the patient;
h) generating a predictive output model set for all outcome variables based on baseline eye tracking metrics and comparing it to reported thresholds for other diseases (either from literature or additional template data sets); show WAB score changes; related eye movements to established markers of another TIA or stroke (predictability or risk of next stroke).
i) rendering information from the report to the caregiver, speech pathologist and assistant associated with the individual/patient.
j) rendering reports to providers for monitoring, flagging any disruptions or changes that may lead to decrease in function or predict another stroke and improvements in health outcomes.

5. Create Personalized and speech therapy stimuli for use in outpatient (in-home) and rehabilitation setting:
a) Content from family to device (And other software)
b) Follow basic stimuli specification as stated in #2.
c) Addition of audio for listening to caregiver/assistant voice.
d) Addition of microphone for patient voice if speech is getting better in outpatient rehabilitation setting.
e) Addition of heartbeat monitor tracking.
f) Addition of fall detection.
g) Provide access to caregiver to add content, increase complexity (with assistance from speech pathologist and assistant).
h) Access to speech pathologist curriculum done weekly for homework. Reiterate the above to generate an optimized model for the individual.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method comprising:
providing a device to an individual, wherein the device comprises a display and an input device, wherein the display provides a set of stimuli and wherein the input device is capable of tracking an eye of the individual;
wherein the input device monitors focus of the individual and wherein the individual's focus on a stimulus in the set of stimuli provides a signal to select that input.

2. The method of claim 1, wherein the selection of the stimulus transmits a request to a caretaker.

3. The method of claim 2, wherein the device is capable of detecting changes in focus which are indicative of a mental state.

4. The method of claim 3, wherein the mental state is selected from depression, anxiety, stress, and fatigue.

5. The method of claim 2, wherein the device is capable of detecting a health event and/or early detection of a health event.

6. The method of claim 5, wherein the health event is selected from stroke and cognitive decline.

7. The device of claim 2, wherein each stimulus in the set of stimuli is displayed as an icon.

8. The device of claim 2, wherein the set of stimuli include at least one of: personal needs, mood, food, and drink.

9. The device of claim 2, wherein at least one stimulus in the set of stimuli represents a hierarchical menu, wherein selection of the at least one stimulus provides a second set of stimuli with more specificity.

10. A device for non-verbal communication comprising:
a display to provide a set of stimuli to an individual; and
an input device capable of tracking an eye of the individual;
wherein the input device monitors focus of the individual and wherein the individual's focus on a stimulus in the set of stimuli provides a signal to select that input.

11. The device of claim 10, further comprising a wireless communication device capable of sending information to another device.

12. The device of claim 10, wherein each stimulus in the set of stimuli is displayed as an icon.

13. The device of claim 10, wherein the set of stimuli include at least one of: personal needs, mood, food, and drink.

14. The device of claim 10, wherein at least one stimulus in the set of stimuli represents a hierarchical menu, wherein selection of the at least one stimulus provides a second set of stimuli with more specificity.

15. A system for non-verbal communication comprising:
a patient device, comprising:
a display to provide a set of stimuli to a patient; and
an input device capable of tracking an eye of the patient;
wherein the input device monitors focus of the individual and wherein the individual's focus on a stimulus in the set of stimuli selects that stimulus; and
a caretaker device, comprising:
a display to provide information to a caretaker; and
an input device capable of accepting input from the caretaker;
wherein a request from a patient is displayed on the display and the caretaker can provide input via the input device to acknowledge a request;
wherein the selection of a stimulus from the patient device sends a request to the caretaker device.

16. The system of claim 15, wherein the patient device and the caretaker device each further comprise a wireless communication device capable of sending and receiving information to each other.

17. The system of claim 15, wherein each stimulus in the set of stimuli is displayed as an icon.

18. The system of claim 15, wherein the set of stimuli include at least one of: personal needs, mood, food, and drink.

19. The system of claim 15, wherein at least one stimulus in the set of stimuli represents a hierarchical menu, wherein selection of the at least one stimulus provides a second set of stimuli with more specificity.

20. The system of claim 15, wherein the caretaker can provide input via the input device to mark a request as complete.
